(19) United States
(12) Patent Application Publication
(10) Pub. No.: US 2018/0360606 A1
(43) Pub. Date: Dec. 20, 2018

(54) FLOW CONTROL VALVES

(71) Applicant: BALATECH LIMITED, Canterbury, Kent (GB)

(72) Inventors: Paul LEONARD, London (GB); Duncan JACKSON, Chicago, IL (US); Eoin BILLINGS, London (GB)

(21) Appl. No.: 16/063,352

(22) PCT Filed: Dec. 21, 2016
(86) PCT No.: PCT/EP2016/082274

§ 371 Date: Jun. 18, 2018

(30) Foreign Application Priority Data
Dec. 21, 2015 (GB) 1522521.2

(51) Int. Cl. A61F 2/24; A61M 27/00; A61M 39/22

(52) U.S. Cl. CPC A61F 2/2475; A61F 2002/48; A61M 39/227; A61M 27/002

(57) ABSTRACT

An implantable controllable fluid flow valve structure for location within an anatomical tube such as a vein or artery, or between apertures in the walls of a vein and artery, is described. The structure has a relatively rigid tubular housing 1 with a tubular elastic cylindrical member 2 within it where the ends of the cylindrical member 2 are attached to the interior wall of the tubular housing 1. The space between the housing wall and the elastic cylindrical member can be increased, for example by pumping fluid 8 under pressure into it, which causes the elastic cylindrical member 2 to distend and thus reduce the flow cross-section of the valve. Mechanical means may be used to effect such distention. The valve structure is particularly useful for use as a valve to control flow through an arteriovenous fistula made surgically, and can also be used as an artificial sphincter.

This invention relates to liquid flow control valves, particularly for implantation in an anatomical tube in humans or animals to control liquid flow through the tube or between two anatomical tubes, in particular to control blood flow through an arteriovenous fistula.

A standard approach to controlling flow through an anatomical tube is to locate adjacent or around the tube a mechanism which may be controlled to squeeze the tube to restrict flow through it. Examples of such devices are disclosed in the following published patent specifications:

WO 86/01395

U.S. Pat. No. 4,408,597

WO 88/05290

EP 2815720

US 2005/0250979

U.S. Pat. No. 4,708,140

US 2011/0306824

US 2004/0138684

US 2012/0095288

US 2011/0071341

WO 99/63907

WO 01/49245

WO 01/10359

Such devices are complex, not easy to install or operate, and are space-consuming.

US 2015/0305746 A1 discloses an arrangement for varying the blood flow in a tube connected between a vein and an artery. The tube incorporates two valves each with an inflatable balloon which when inflated restricts the blood flow in the tube. The arrangement is complex and the valve disclosed in this specification is liable to give rise to a substantial risk of blood clotting either in the region of the valve itself or in the associated implantable graft.

According to the present invention, there is provided an implantable controllable fluid flow valve structure comprising a relatively rigid tubular housing of diameter suitable for location within the lumen of an anatomical tube, the housing having fixed within it a tubular elastic cylindrical member, the ends of the cylindrical member being secured to the interior wall of the tubular housing at two or more axially spaced positions, and means for distending the elastic member to cause portions of it between the two axially spaced locations where it is attached to the relatively rigid tubular member to move radially inwardly whereby to restrict axial liquid flow through the valve structure, and means for attaching the valve structure within the anatomical tube or to apertures formed in the walls of two anatomical tubes.

In a first preferred embodiment, the movement of the elastic cylindrical member may be achieved by increasing the pressure in a hydraulic fluid between the outer wall of the cylindrical elastic member and the inner wall of the tubular housing. In an alternative, the annular space between the external wall of the elastic member and the internal wall of the relatively rigid tubular housing may have one or more cords passing through it where increasing the tension on the cord or cords causes part of the elastic member to distend inwardly away from the inner wall of the housing and thus reducing the size of the flow passage through the valve structure. Relieving that tension allows the elastic member to spring back with consequent increase in the cross-section of the flow passage. In another alternative, the valve may be actuated by a pump and reservoir arrangement.

In either case, there is an aperture in the wall of the relatively rigid tubular housing through which hydraulic fluid may be passed into the space between the relatively rigid outer tubular housing and the elastic tubular member or withdrawn from it, or through which one or more cords or wires may pass which may be pulled to reduce the size of the flow passage by causing the portions of the elastic member to move away from the wall of the relatively tubular member or which can allow the passage to open up again due to the elastic nature of the inner tubular member when the tension is released.

Actuation of the valve may be achieved by using a simple plunger mechanism when the valve is hydraulically operated or a Bowden cable type arrangement where movement is achieved by one or more cords.

In either case, the extent of closing of the valve, i.e. the extent of restriction of the axial flow path through it, may be conveniently adjusted by means of a screw-threaded mechanism. Turning of a rotatable disc fitted to a threaded plunger in a cylinder filled with hydraulic fluid and connected to the space between the outer housing and the cylindrical elastic member may enable fine adjustment of the degree of opening or closing of the valve.

In the case where the valve is to be used to control liquid flow through a tubular anatomical passage, such as a vein or artery or through an arteriovenous fistula, the entire valve and its actuation mechanism may be designed to be subcutaneously implantable, with the rotatable disc just mentioned movable relative to some form of housing by magnetic means. In a particularly preferred embodiment, a disc with magnetised sectors is located in a housing having a relatively flat surface which is located below the skin of a human or animal, and where rotation of the disc causes a screw-threaded member to be moved axially relative to the disc in the housing, either to compress or reduce the pressure in a hydraulic fluid inside the housing or to move one or more cords to achieve the desired valve opening or closing effect.

Valves according to the invention may be made of a wide variety of materials and on a wide variety of scales suitable for the intended purpose. For medical purposes where it is desired to control the flow of fluid through an anatomical tubular vessel, the entire unit may be made of appropriately biocompatible polymeric material forming a casing and where the elastic cylindrical member is likewise made of biocompatible elastic polymeric material.

The cross-section of the relatively rigid exterior support tube may be circular or, for example, oval or elliptical. The shape of the support tube, and the shape of both the undistended and distended elastic cylindrical member should be chosen to minimise turbulence in the liquid flowing through the valve, as should the contour of the ends of the elastic cylindrical member where they join or merge into the wall of the exterior support tube.

The axial extent of the support tube and the elastic cylindrical member may vary widely. The ratio of length to diameter of the elastic cylindrical member is preferably between 0.5 and 2.5. The diameter of the support tube will depend on the anatomical tube into which it is to be inserted, For use in arteries or veins, or for use in an arteriovenous fistula between brachial artery and cephalic vein (standard in preparation for dialysis in renal disease patients) a diameter of 4 to 5 mm is often appropriate.

Valves in accordance with the present invention essentially function analogously to an anatomical sphincter, but are customarily open for fluid flow through them and closed when desired rather than the normal operation of sphincter valves in the human or animal body which are normally closed and then opened by appropriate musculature on voluntary or involuntary command by the animal or human in question. Valves according to the invention may be used to function as an artificial sphincter in any part of the body, for example the urinary sphincter, the ileocaecal sphincter, the anal sphincter. With suitable design, they may be used to control flow of materials other than bodily liquids, and over a wide range of viscosities, both for simple liquids or fluids, as well as other flowable mixes such as suspended solids in a liquid or gaseous medium.

The valves in accordance with the present invention are of particular value in providing a simple valve which can be used to control blood flow through an arteriovenous fistula. The use of a valve to control blood flow through an arteriovenous fistula is disclosed in WO 2015/135955 A2. However, the disc or flap valve constructions there-described may be susceptible in use to malfunction due to the build-up of deposits. The valve in accordance with the present invention provides a much smoother flow pattern, either in fully open condition when the elastic cylindrical member lies essentially close to the wall of the relatively rigid tubular member and when the valve is closed or the flow path restricted by external radially inward forces generated, for example, by the hydraulic or mechanical mechanisms described above.

A particular advantage of the use of valves in accordance with the present invention to control blood flow through an arteriovenous fistula is that the flow rate can be adjusted by a physician or other medical operative such as a nurse, to a desired flow appropriate for the patient concerned, for example a patient undergoing haemodialysis as described in more detail in the international publication referred to above.

The invention is illustrated by way of example with reference to the accompanying drawings in which:

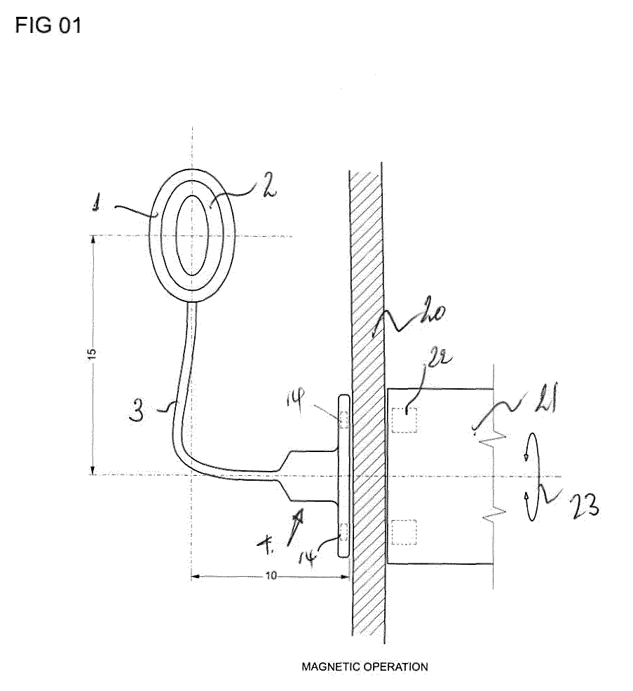

FIG. 1 is a diagrammatic illustration of a valve in accordance with the present invention and means for actuating it;

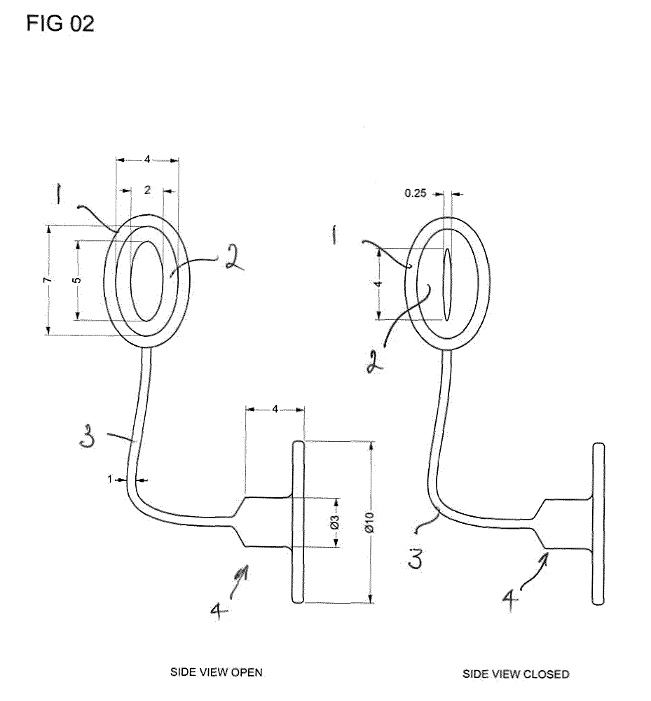

FIG. 2 are diagrams illustrating the possibility of different flow rates through the valve;

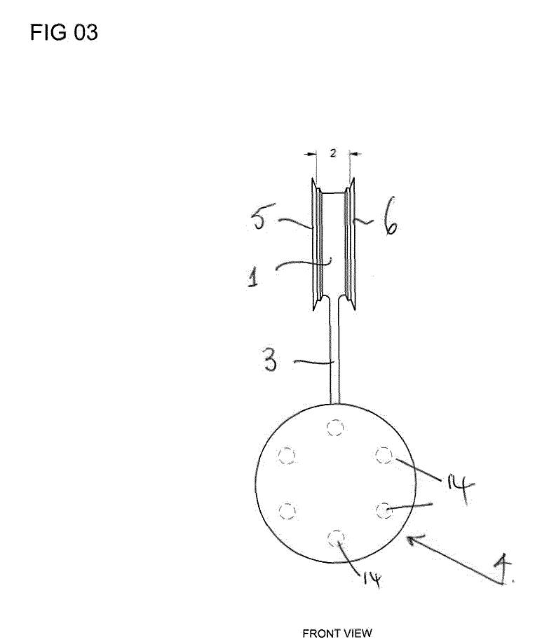

FIG. 3 is a diagrammatic perspective view of an implantable arteriovenous fistula valve in accordance with the present invention;

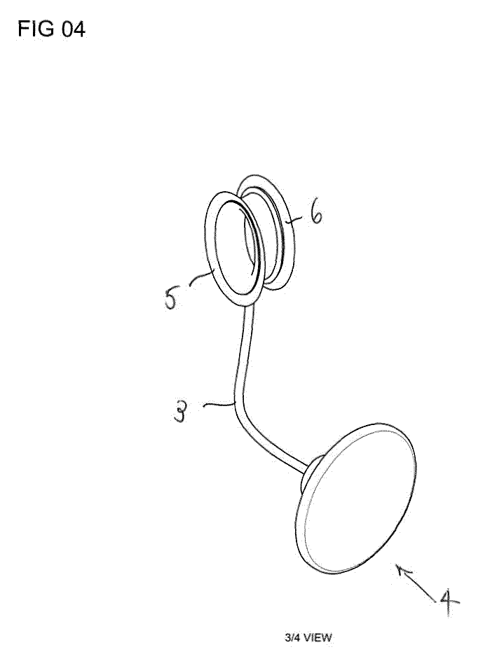

FIG. 4 is a front view of the valve shown in FIG. 3;

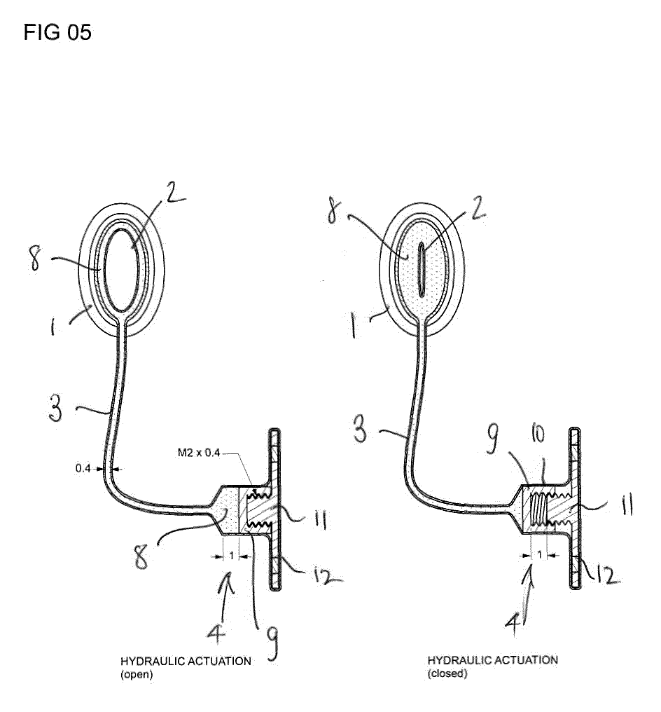

FIG. 5 shows the mode of operation of a hydraulically adjusted valve;

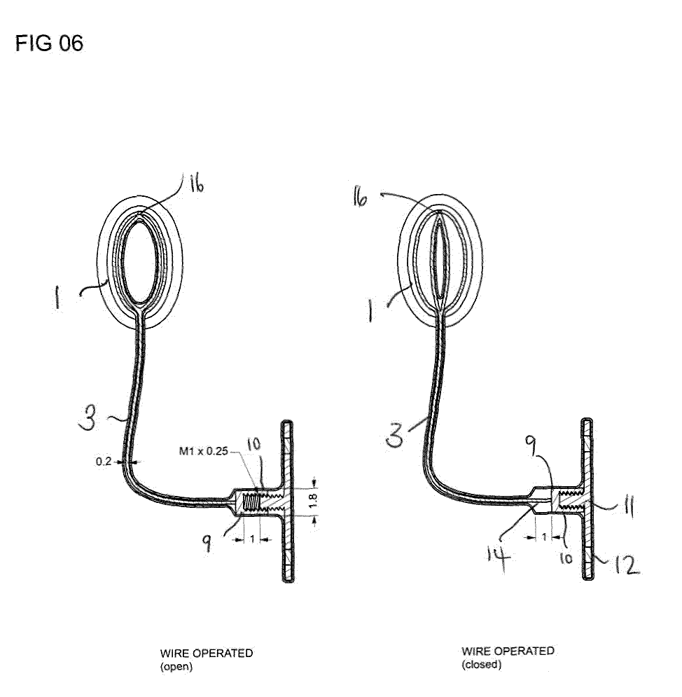

FIG. 6 shows the mode of operation of a wire adjusted valve;

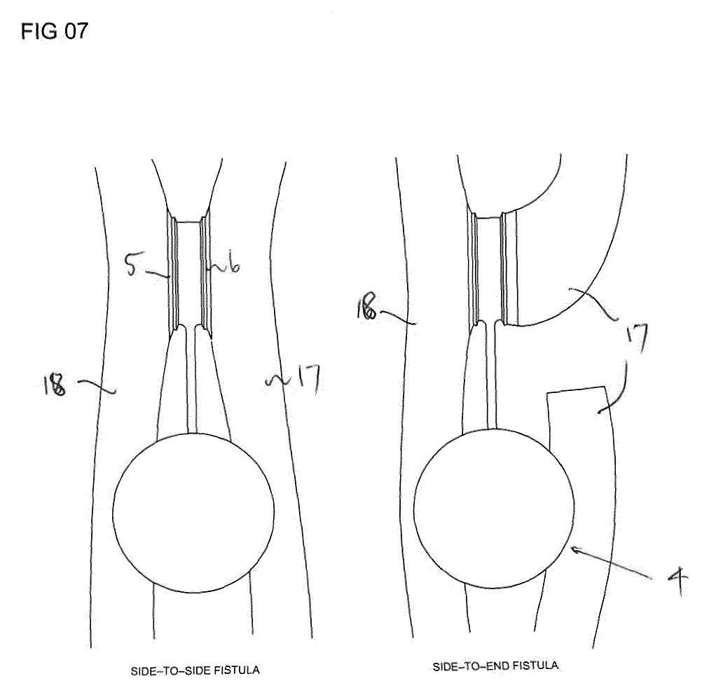

FIG. 7 shows diagrammatically the installation of the valve shown in FIGS. 3 and 4 following surgical implantation;

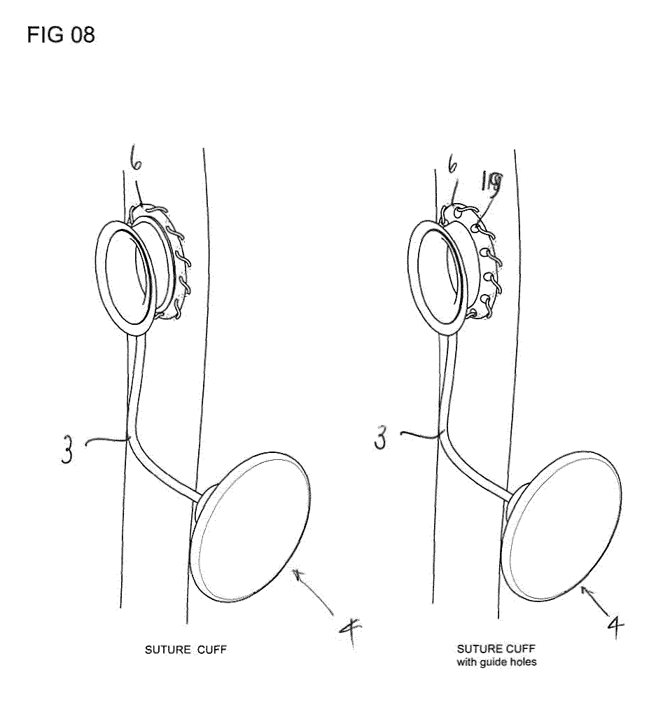

FIG. 8 shows detail of how the valve is attached to an arterial or venous wall;

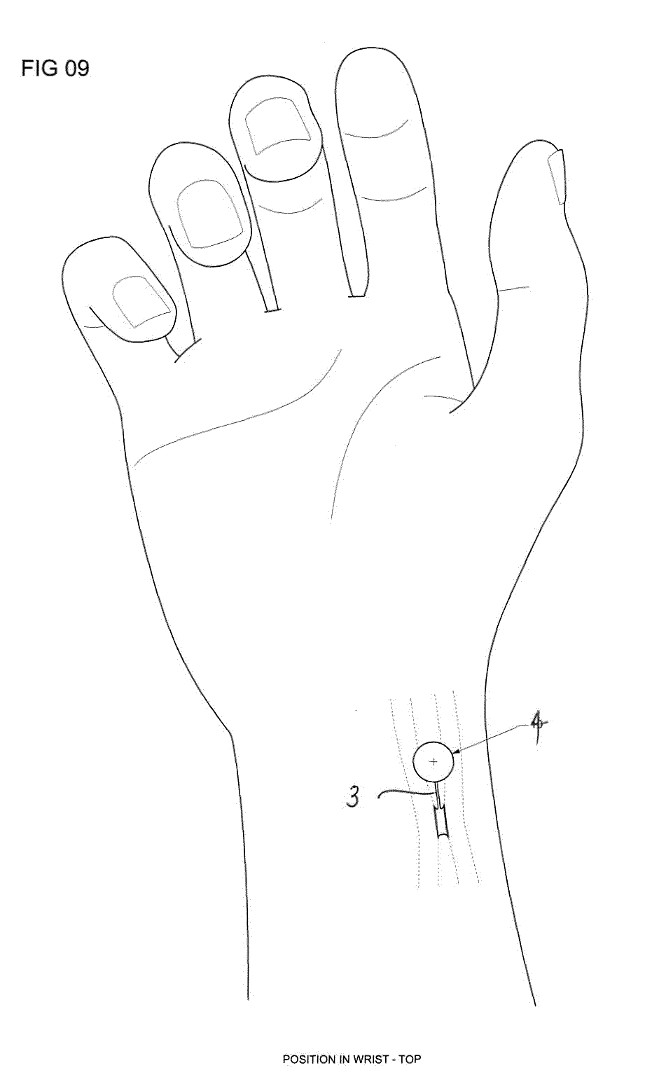

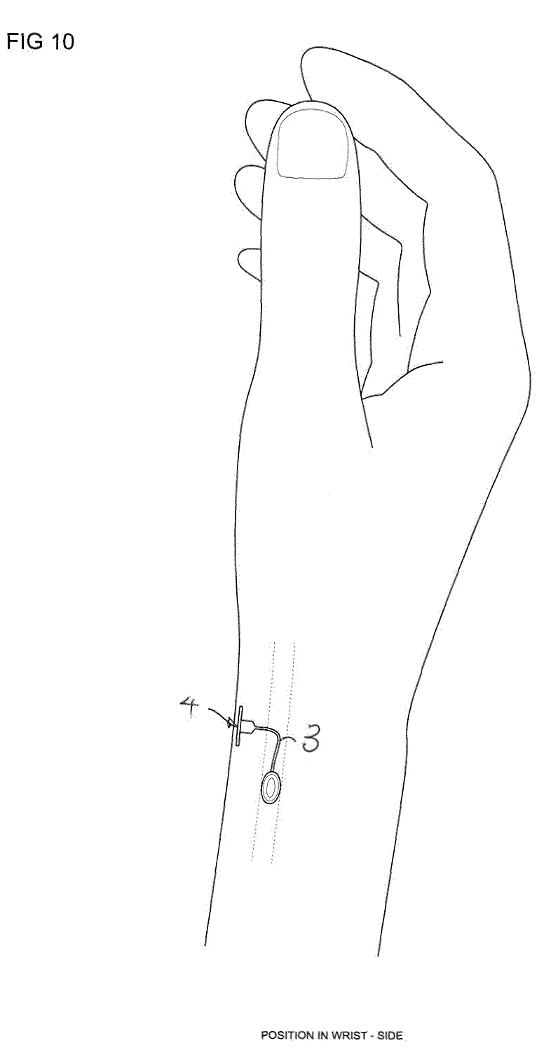

FIGS. 9 and 10 are diagrammatic views of the installation of the valve controlling the flow through an arteriovenous fistula in a patient's wrist; and

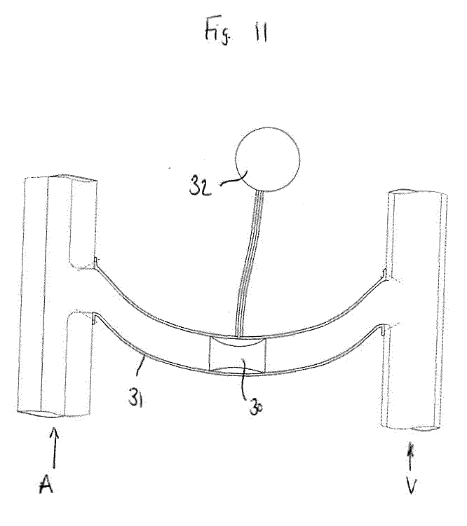

FIG. 11 is a diagrammatic illustration of the use of the valve according to the invention in a tube connected between a vein and an artery.

In the drawings, all dimensions indicated are in millimetres.

Referring to the drawings, these show a valve structure in accordance with the present invention configured as a valve to control the flow through an arteriovenous fistula. The valve structure itself has an outer relatively rigid tubular housing 1 and located within it a cylindrical elastic membrane 2, the circular edges of the ends of membrane 2 being attached in fluid tight fashion to the interior wall of member 1. As shown, the overall shape of the valve structure is somewhat elliptical rather than circular. The valve is operated to control fluid flow via a connector member 3 by an actuator unit 4.

The actuator unit 4 consists of a housing having a generally cylindrical portion defined by an outer wall 10 and located within the cylindrical portion of wall 10 is a piston or thrust member 9. The position of piston or thrust member 9 is controlled by rotation of a disc 11 having a threaded stud protruding from one side. Turning disc 11 causes the piston or thrust member 9 to move to the left or right as seen in FIGS. 5 and 6. By moving the piston, the membrane 2 may be distended radially relative to the housing 1 to reduce the flow passage through the valve structure as shown in the right-hand portion of FIG. 2 or to increase the flow cross-section, for example to that shown in the left-hand side of FIG. 2.

Rotation of the disc 11 with its threaded stud is achieved by means of a handle diagrammatically indicated as 21 in FIG. 2 which has a circular cross-section and, set into its front end, a set of magnets 22. These cooperate with a set of magnets 14 set into the rotatable disc 11 so that if the handle 21 is placed adjacent the disc 11, which is in the housing of actuator unit 4, and rotated as shown by the double-headed arrow 23 in FIG. 1, the disc 11 with its threaded stud rotates and moves the piston or thrust member 9 to the left or right depending upon whether handle 21 is turned clockwise or anti-clockwise.

Two embodiments are shown in the drawings, in one of which the degree of opening of the valve is controlled by hydraulic pressure and in the other by a mechanical wire construction.

The first of these is diagrammatically illustrated in FIG. 5 where the connector member 3 is a tube filled with liquid 8 which fills the entirety of tube 3, the space between the elastic membrane 2 and the outer housing 1 and the generally cylindrical chamber in the actuation head 4. As can be seen in FIG. 5, rotating disc 11 so that the piston 9 moves to the left urges the membrane 2 to distend, thus narrowing the passage through the valve structure. Moving the piston 9 to the right allows the membrane 2 to relax and come to lie adjacent the walls of the relatively rigid outer member 1 as shown on the left-hand side of FIG. 5. The liquid 8 should be biocompatible so that any leakage has no deleterious effect, for example artificial blood or blood plasma.

FIG. 6 shows an alternative construction where the member 9 is a thrust member and which is connected to a wire or cord 14 which passes through the connector tube 3. The far end of the wire or cord 14 is connected at 16 to the interior wall of the relatively rigid external member 1 forming part of the valve structure itself. As shown on the right-hand side of FIG. 6, when the thrust member 9 is pulled to a position as far to the right as it will go, wire or cord 14 is under tension and causes the membrane 2 to distend to reduce the flow through the valve. If the thrust member 9 is moved to the left as shown on the left-hand side of FIG. 6, then the valve is opened for maximum fluid flow through it.

As noted above, the valve diagrammatically illustrated is useful for controlling blood flow through an arteriovenous fistula surgically created between an artery 17 and a vein 18 as shown in FIG. 7. The ends of the relatively rigid housing have flanges 5 and 6 on them which may be sutured around an aperture formed in the artery or vein wall directly if the flange 5, 6 is made of appropriate material or via a set of holes 19 in flanges 5 and 6 otherwise, as shown on the right in FIG. 8.

A preferred site for an arteriovenous fistula used in surgery to provide for renal dialysis purposes, as described in the international publication referred to above, is in a patient's wrist. FIGS. 9 and 10 show how the unit may be installed. The actuation head 4 lies under the skin and the disc within it may be rotated using the tool 21 shown in FIG. 1. In that Figure, the skin is diagrammatically illustrated by a hatched wall 20.

FIG. 11 shows an alternative approach, of particular value where it is desired to provide blood flow between an artery and a vein, but where it is not desired, during surgery to make a connection between artery and vein, to relocate either from its usual anatomical position. As shown diagrammatically, the valve of the invention, denoted 30, is incorporated in a tube 31 which is sutured at its ends 33 in standard fashion around the periphery of an aperture in the wall of the vein V or artery A. The actuation of the valve 30 is by way of a mechanism 32 as described above.

1-11. (canceled)

12. An implantable controllable fluid flow valve structure comprising a relatively rigid tubular housing of diameter suitable for location within the lumen of an anatomical tube, the housing having fixed within it a tubular elastic cylindrical member, the ends of the cylindrical member being secured to the interior wall of the tubular housing at two or more axially spaced positions, and means for distending the elastic member to cause portions of it between the two axially spaced locations where it is attached to the relatively rigid tubular member to move radially inwardly whereby to restrict axial liquid flow through the valve structure, and means for attaching the valve structure within the anatomical tube or to apertures formed in the walls of two anatomical tubes.

13. A valve structure according to claim 12 wherein the movement of the elastic cylindrical member is achieved by increasing the pressure in a hydraulic fluid between the outer wall of the cylindrical elastic member and the inner wall of the tubular housing.

14. A valve structure according to claim 12 wherein the annular space between the external wall of the elastic member and the internal wall of the relatively rigid tubular housing has one or more cords passing through it, and means are provided to increase the tension on the cord or cords so as to cause part of the elastic member to distend inwardly away from the inner wall of the housing so as to reduce the size of the flow passage through the valve structure.

15. A valve structure according to claim 13 further comprising an aperture in the wall of the relatively rigid tubular housing through which hydraulic fluid may be passed into the space between the relatively rigid outer tubular housing and the elastic tubular member or withdrawn from it, or through which one or more cords may pass which may be pulled to reduce the size of the flow passage by causing the portions of the elastic member to move away from the wall of the relatively tubular member or which can allow the passage to open up again due to the elastic nature of the inner tubular member when the tension is released.

16. A valve structure according to claim 14 further comprising an aperture in the wall of the relatively rigid tubular housing through which hydraulic fluid may be passed into the space between the relatively rigid outer tubular housing and the elastic tubular member or withdrawn from it, or through which one or more cords may pass which may be pulled to reduce the size of the flow passage by causing the portions of the elastic member to move away from the wall of the relatively tubular member or which can allow the passage to open up again due to the elastic nature of the inner tubular member when the tension is released.

17. A valve and actuator assembly comprising a valve structure according to claim 12 and a plunger mechanism when the valve is hydraulically operated or a Bowden cable type arrangement where movement is achieved by one or more cords.

18. An assembly according to claim 17 wherein the extent of restriction of the axial flow path through the valve may be adjusted by means of a screw-threaded mechanism.

19. An assembly according to claim 18 wherein the screw-threaded mechanism includes a plunger to enable fine adjustment of the degree of opening or closing of the valve.

20. An assembly according to claim 17 adapted to control liquid flow through a tubular anatomical passage.

21. An assembly according to claim 20 wherein the entire valve structure and its actuation mechanism are subcutaneously implantable and the screw-threaded mechanism is movable by magnetic means.

22. An assembly according to claim 21 and including a rotatable disc having magnetised sectors located in a housing having a relatively flat surface and where rotation of the disc causes a screw-threaded member to be moved axially relative to the disc in the housing, either to compress or reduce the pressure in a hydraulic fluid inside the housing or to move one or more cords to achieve the desired valve opening or closing effect.

23. An assembly according to claim 17 further comprising a casing of biocompatible polymeric material and wherein the elastic cylindrical member is also made of biocompatible elastic polymeric material.
